(19) United States
(12) Patent Application Publication
(10) Pub. No.: US 2004/0028552 A1
(43) Pub. Date: Feb. 12, 2004

(54) Gas contact ultrasound germicide and therapeutic treatment

(76) Inventors: Mahesh C. Bhardwaj, State College, PA (US); Kelli Hoover, Pennsylvania Furnace, PA (US); Nancy Ostiguy, State College, PA (US)

Correspondence Address: David C. Hanson, Attorney for Applicants, 700 Koppers Building, 436 Seventh Avenue, Pittsburgh, PA, 15219-1818, US

(21) Appl. No.: 10/393,128

(22) Filed: Mar. 20, 2003

Related U.S. Application Data
(60) Provisional application No. 60/365,927, filed on Mar. 20, 2002.

(51) Int. Cl. A61L 2/025

(52) U.S. Cl. 422/20

(57) ABSTRACT

A method for destroying bacterial spores, germs, and the like is disclosed, comprised of a non-contact ultrasound device that produces ultrasonic beams transferred in ambient air without the use of a liquid medium in frequencies ranging between 50 kHz to 5 MHz. The method of the present invention can be used as a germicide for destroying anthrax spores and other highly toxic microbes that may be used as an agent of bioterrorism.

### CROSS REFERENCE TO RELATED APPLICATION

This application claims the benefit of U.S. Provisional Patent Application No. 60/365,927, filed Mar. 20, 2002, which is incorporated herein by reference.

### BACKGROUND OF THE INVENTION

Disease-causing microorganisms can be highly resistant to killing and can exhibit high toxicity in low numbers, making it difficult to control human exposure through air-delivered mechanisms. For example, bioterrorism in the form of aerosolized anthrax spores (Bacillus anthracis) passing through the mail presents a serious challenge for the postal services. Technologies currently available to accomplish decontamination of microbes have significant limitations, in part because organisms like Bacillus spores, protozoan cysts, and some viruses are resistant to drying, heat, ultraviolet light, gamma radiation, and many disinfectants.

Some examples of currently available sterilization methods that have been used for many years include autoclave sterilization, which involves using steam under pressure. The high, wet heat destroys many pathogenic bacteria and the high pressure increases the efficacy of this process. However, steam cannot be used for many applications, including the killing of pathogens in/on mail, because the material being sterilized does not tolerate being wet or placed in a chamber at high pressure.

Ethylene oxide is another effective but limited sterilization method. Ethylene oxide is compatible with most medical materials, especially those that are heat sensitive. This method, however, has several limitations, such as the need to pre-treat materials with high levels of humidity in order to increase penetration of the ethylene oxide; to treat the materials for several hours; and to aerate the treated materials for several hours to several days to allow for the off-gassing of the ethylene oxide. Moreover, ethylene oxide is flammable, explosive, a toxic air contaminant, an ozone depleter, and is a probable human carcinogen.

Electron beam sterilization is a non-radioactive sterilization method using electrons fired from a cathode that can be used to kill anthrax and other pathogens. The high-energy electron stream sterilizes by sweeping an object in a shielded chamber and breaking the chemical bonds of organic compounds to produce very reactive free radicals. The depth of beam penetration is inversely proportional to the density of the material being treated. For effective treatment, however, relatively long exposure periods are necessary and penetration of packages is difficult. In addition, shielding is necessary to protect workers from the radiation formed during the production of the electron beam.

Gamma radiation, from sources such as Cobalt 60 and X-rays, has been used for many years to reduce the number of pathogens in food products, and can be used to destroy anthrax spores. Both types of radiation can penetrate envelopes and packages and irradiated material does not become radioactive and can be handled safely by postal employees and the public after treatment. However, the major disadvantages of gamma radiation are the relatively long exposure times needed for effective sterilization and the need for substantial shielding between workers and the radiation source.

Two other methods include ultraviolet light, which can be used to inactivate microbes on surfaces, but has very limited penetration ability, and sonication (high power low frequency ultrasound with water as the wave carrier medium), which has been shown to destroy bacterial spores. For obvious reasons, however, sonication would not be useful for applications in which contact with the material to be treated is impractical, such as where unwanted germs need to be destroyed under ambient or closed environments composed of air or other gases.

Since its first practical use for detection of underwater objects, non-destructive and non-invasive applications of ultrasound have advanced significantly. Low power ultrasound is widely used for non-destructive evaluation of industrial materials for defect, microstructure, and property characterization, as well as in medical diagnostics for fetus development and tissue analysis. High power ultrasound has many uses including, but not limited to, cell disruption, particle size reduction, welding, and vaporization. It is being further developed for chemical reaction acceleration, invasive and non-invasive therapeutics, surgical procedures, and levitation.

A common denominator of all conventional applications of ultrasound is that the ultrasound source—the transducer—is physically coupled, either directly or indirectly, to the medium to be tested or treated. Generally, the coupling agents are liquids, such as water, oils, gels, or grease. Physical coupling has been necessary in order to efficiently transmit ultrasound in the materials. Ultrasound transmitted through air has been used to remove spores from an object so that they may be exposed to ultraviolet light and thus destroyed. (See Rose et al. U.S. Pat. No. 6,090,346, issued Jul. 18, 2000, entitled “Sterilization Using Ultraviolet Light and Ultrasonic Waves”.) However, it has not been considered possible to use ultrasound alone to destroy bacteria and spores.

Thus, there is a need for an effective, safe and efficient methodology to accomplish decontamination of microbes and other pathogens without the significant limitations inherent in the currently available methods.

### SUMMARY OF THE INVENTION

Briefly, according to the present invention, there is provided a method for destroying bacterial spores, germs, and the like, such as anthrax, comprising a non-contact ultrasound device that produces planar, point, or cylindrically focused ultrasonic beams in a frequency range between 50 kHz to 5 MHz, wherein the beams are transferred in gas or ambient air without the use of a liquid medium. The ultrasonic device may be separated from the material to be treated by a distance between 1.0 mm to 7.0 mm and can be excited by about 1000 bursts to 2000 bursts of about 0.10 V to 1.0 V that is augmented with a power amplifier of about 50 dB to 55 dB with a pulse frequency of about 100 ms to 500 ms lasting less than 1 to 5 minutes. Alternatively, the ultrasonic device may be excited in a continuous wave mode having an amplitude of about 0.1 V that increases to 0.2 V after less than 1 to 3 minutes and that lasts 10 to 20 minutes. The method of the present invention has the potential to be used as a general germicide for the sterilization of medical and surgical equipment and food materials, in addition to destruction of anthrax spores and other highly toxic microbes that may be used as an agent of bioterrorism. Furthermore, there is the potential to use this method in large-scale situations, such as in decontamination of air duct systems of buildings, airplanes, and space stations.

### BRIEF DESCRIPTION OF THE DRAWINGS

FIG. 1 illustrates an experimental setup for this invention; and

FIGS. 2(a) and 2(b) are digital images of bacterial cultures in petri dishes which illustrate the effectiveness of this invention.

### DESCRIPTION OF THE PREFERRED EMBODIMENTS

The present invention includes a method for destroying unwanted germs and tissues, such as biohazard materials, bacterial spores, viruses, and other hazardous biological and chemical materials, using an ultrasonic transducer, for example, according to U.S. Pat. No. 6,311,573, incorporated herein by reference, which is characterized by extremely high transduction in air or other gases (either under ambient environment or high pressures). For example, use of the ultrasound transducer as described in the present invention destroys 99.9% of dried bacterial spores of a close relative of anthrax, Bacillus thuringiensis, without direct or indirect contact between the transducer and the material to be treated. The invention of such non-contact ultrasound (NCU) transducers opens the door for significant applications in biomedical, food safety, environmental safety, and other fields.

The efficiency of an ultrasonic transducer is dependent on the coupling coefficients and other electromechanical properties of the piezoelectric material. It also depends upon the mechanism by which ultrasound is transferred from the piezoelectric material to the medium in which ultrasound needs to be propagated. In the non-contact mode, this medium is air. Since the acoustic impedances of piezoelectric materials are several orders of magnitude higher than that of air, it is usually necessary to implant transitional (acoustic impedance matching) layers of various materials in front of the piezoelectric material. Ultimately, it is the characteristics of the final layer that determine the transduction efficiency of a transducer device.

Based upon compressed fiber as the final matching layer, the NCU transducers used in the present invention are capable of efficiently transmitting ultrasound into air from the matching layer and can generate immense acoustic pressures in ambient air. For example, pressures from 20 Pa/V to 150 Pa/V have been observed between 100 kHz to 4 MHz frequencies. Despite the high attenuation of ultrasound by air, these acoustic pressures are substantial, though smaller than for similar transducers in water, which can typically range from 1 Kpa/V to 5 Kpa/V.

The method disclosed in the present invention comprises using a high power generating non-contact ultrasonic transducer that produces planar, point, or cylindrically focused ultrasonic beams that can be transferred in gas or ambient air without the use of a liquid medium. The non-contact ultrasonic transducer is separated from the material to be treated, which includes, without limitation, bacterial spores, viruses, and other pathogens, by a distance of preferably 1.0 mm to 7.0 mm, more preferably 2.0 mm to 6.0 mm, and most preferably 3.0 mm to 5.0 mm. The material to be treated is then irradiated in a frequency range of preferably 50 kHz to 5 MHz, more preferably 85 kHz to 185 kHz, and most preferably 100 kHz to 161 kHz. The non-contact ultrasonic transducer can be excited in a pulsed mode by about 1000 bursts to 2000 bursts of preferably 0.10 V to 1.0 V, more preferably 0.25 V to 1.0 V, and most preferably 0.5 V to 0.7 V, excitation levels being augmented with a power amplifier of about 50 dB to 55 dB. The pulse repetition frequency of excitation may range between 100 to 500 milliseconds and may last about 1 to 5 minutes. Alternatively, the non-contact ultrasonic transducer may be excited in a continuous wave mode with an amplitude of about 0.1 V that increases to about 0.2 V after less than 1 to 3 minutes, excitation augmented with a power amplifier of about 50 dB to 55 dB and lasting about 10 to 20 minutes.

Eliminating contact between the ultrasound transducer and the material to be treated facilitates, without limitation: (1) the analysis of green, unpolymerized, liquid-sensitive, porous, and other materials, or when contact is simply a nuisance; (2) non-invasive medical diagnostics when contact with a patient is harmful or painful; (3) destruction of unwanted germs in the environment or containers and surface and subsurface treatment of wounds, scars, malignant tissue, etc.; sterilization of medical, food, and pharmaceutical components and equipment; and disinfection and decontamination of foods.

The present invention can be described by the following experimental design, which is intended to be illustrative only, since numerous modifications and variations of the parameters disclosed will be apparent to those skilled in the art.

Experimental Design—Destruction Of Bacterial Spores By High Efficiency NCU Transducers

1. Introduction

To determine the feasibility of NCU transducers to kill bacterial spores, a series of experiments were conducted in which lyophilized (freeze-dried) Bt spores were irradiated with NCU transducers in the frequency range of 50 kHz to 5 MHz, varying the exposure time from 1 to 20 minutes. Bt is considered a good model for Bacillus anthracis (anthrax). Bacillus thuringiensis, Bacillus anthracis, and Bacillus cereus are very close relatives; in fact some researchers consider them to belong to the same species. (Helgason, E. et al., Bacillus anthracis, Bacillus cereus, and Bacillus thuringiensis—one species on the basis of genetic evidence. Applied and Environmental Microbiology, 66: 2627-2730, 2000).

Both Bacillus anthracis and Bacillus cereus are opportunistic human pathogens. Thus, it is safer to work with a close relative that is benign to humans, such as Bacillus thuringiensis (Bt). Bt is known to be a useful source of insecticidal toxins, often in the form of spore-containing preparations of crystal proteins that are spread from airplanes over fields to kill insects. The three bacilli differ by only a few genes on their plasmids (extra-chromosomal DNA) that encode different toxins. If you remove the plasmids from Bt, the bacillus cannot be distinguished from either Bacillus anthracis or Bacillus cereus. Thus, Bt can be used as a safe model for testing methods to kill the spores of Bacillus anthracis without having to risk working directly with the deadly bacterium. Indeed, to work directly with Bacillus anthracis requires a biosafety level 3 facility.

2. Material And Methods

In the experimental procedure described herein, two NCU transducers, according to U.S. Pat. No. 6,311,573, with pulsed and continuous wave (CW) modes of transducer excitation were used. The NCU transducers had the following specifications:

| | Transducer #1 | Transducer #2 |
|---|---|---|
| Nominal frequency (kHz) | 200 | 100 |
| Active area diameter (mm) | 50 | 50 |
| Loop sensitivity in air (dB) | −40 | −54 |
| Acoustic power (Pa/V) | approximately 60 | approximately 25 |

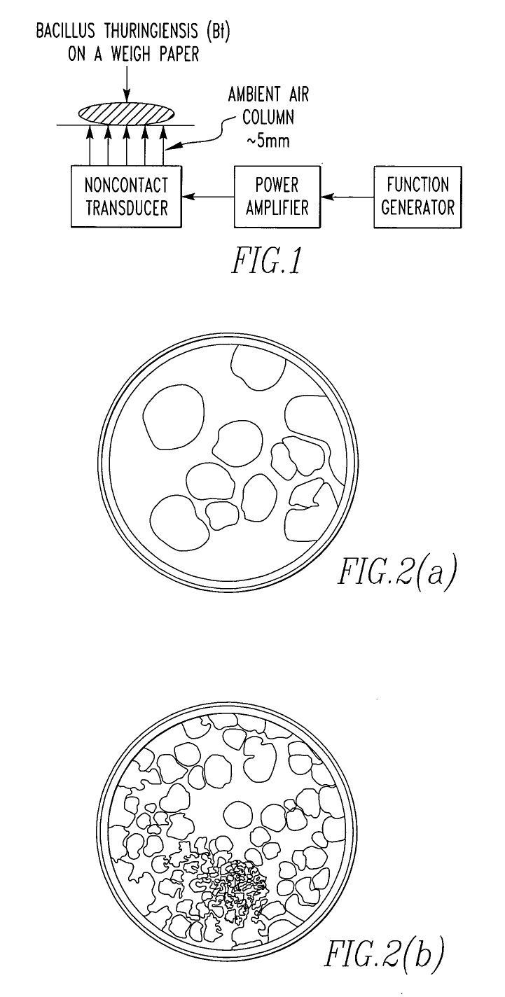

Both transducers were excited using an appropriate sinusoidal burst or continuous wave (CW) signal from a function generator amplified by 55 dB by a power amplifier. A schematic of the experimental design is shown in FIG. 1.

Spores of Bt were grown in LB broth in a 2 ml Ehrlenmeier flask at 28° C. on a shaking incubator until sufficient sporulation had occurred (approximately 2 weeks). The spores were washed several times in sterile milliQ water and then lyophilized overnight. Small amounts of each test sample of lyophilized spores was placed on a thin glossy piece of paper, which was then placed approximately 3 to 4 mm away from the active area of the NCU transducer and subjected to different NCU treatment parameters for various periods of time in an attempt to kill the spores (see Table 1 for treatment parameters). Each group of spores received only one type of treatment as outlined in Table 1 on the following page.

TABLE 1

| | | Colony | | | | | | |
|---|---|---|---|---|---|---|---|---|
| Packet | Dilution | # | CFU | CFU/ml | % inactivation | Fold Decrease | Time | Treatment |
| p29 | 1.00E−06 | 23 | 2.30E+07 | 5.75E+07 | 99.0417% | 104.35 | 10 sec | F = 93 PRF = 50 Burst = 2000 |
| | | | | | | | | DTY = 50% Amp = 0.5 Offset = 0 |
| p30 | 1.00E−06 | 31 | 3.10E+07 | 7.75E+07 | 98.7083% | 77.42 | 10 sec | F = 93 PRF = 50 Burst = 2000 |
| | | | | | | | | DTY = 50% Amp = 0.5 Offset = 0 |
| p31 | 1.00E−06 | 3 | 3.00E+06 | 7.50E+06 | 99.8750% | 800.00 | 10 sec | F = 93 PRF = 50 Burst = 2000 |
| | | | | | | | | DTY = 50% Amp = 0.5 Offset = 0 |
| p32 | 1.00E−04 | 56 | 5.60E+05 | 1.40E+06 | 99.9767% | 4285.71 | 30 sec | F = 93 PRF = 50 Burst = 2000 |
| | | | | | | | | DTY = 50% Amp = 0.5 Offset = 0 |
| p33 | 1.00E−04 | 73 | 7.30E+05 | 1.83E+06 | 99.9696% | 3287.67 | 30 sec | F = 93 PRF = 50 Burst = 2000 |
| | | | | | | | | DTY = 50% Amp = 0.5 Offset = 0 |
| p34 | 1.00E−04 | 34 | 3.40E+05 | 8.50E+05 | 99.9858% | 7058.82 | 30 sec | F = 93 PRF = 50 Burst = 2000 |
| | | | | | | | | DTY = 50% Amp = 0.5 Offset = 0 |
| p35 | 1.00E−04 | 19 | 1.90E+05 | 4.75E+05 | 99.9921% | 12631.58 | 60 sec | F = 93 PRF = 50 Burst = 2000 |
| | | | | | | | | DTY = 50% Amp = 0.5 Offset = 0 |
| p36 | 1.00E−04 | 71 | 7.10E+05 | 1.78E+06 | 99.9704% | 3380.28 | 60 sec | F = 93 PRF = 50 Burst = 2000 |
| | | | | | | | | DTY = 50% Amp = 0.5 Offset = 0 |
| p37 | 1.00E−04 | 67 | 6.70E+05 | 1.68E+06 | 99.9721% | 3582.09 | 60 sec | F = 93 PRF = 50 Burst = 2000 |
| | | | | | | | | DTY = 50% Amp = 0.5 Offset = 0 |
| p38 | 1.00E−06 | 89 | 8.90E+07 | 2.23E+08 | 96.2917% | 26.97 | 180 sec | F = 93 PRF = 50 Burst = 2000 |
| | | | | | | | | DTY = 50% Amp = 0.5 Offset = 0 |
| p39 | 1.00E−06 | 12 | 1.20E+07 | 3.00E+07 | 99.5000% | 200.00 | 180 sec | F = 93 PRF = 50 Burst = 2000 |
| | | | | | | | | DTY = 50% Amp = 0.5 Offset = 0 |
| p40 | 1.00E−06 | 9 | 9.00E+06 | 2.25E+07 | 99.6250% | 266.67 | 180 sec | F = 93 PRF = 50 Burst = 2000 |
| | | | | | | | | DTY = 50% Amp = 0.5 Offset = 0 |
| p41 | 1.00E−06 | 26 | 2.60E+07 | 6.50E+07 | 98.9167% | 92.31 | 10 sec | F = 161 PRF = 100 Burst = 2000 |
| | | | | | | | | DTY = 50% Amp = 0.5 Offset = 0 |
| p42 | 1.00E−06 | 23 | 2.30E+07 | 5.75E+07 | 99.0417% | 104.35 | 10 sec | F = 161 PRF = 100 Burst = 2000 |
| | | | | | | | | DTY = 50% Amp = 0.5 Offset = 0 |
| p43 | 1.00E−06 | 36 | 3.60E+07 | 9.00E+07 | 98.5000% | 66.67 | 10 sec | F = 161 PRF = 100 Burst = 2000 |
| | | | | | | | | DTY = 50% Amp = 0.5 Offset = 0 |
| p44 | 1.00E−06 | 18 | 1.80E+07 | 4.50E+07 | 99.2500% | 133.33 | 30 sec | F = 161 PRF = 100 Burst = 2000 |
| | | | | | | | | DTY = 50% Amp = 0.5 Offset = 0 |
| p45 | 1.00E−06 | 21 | 2.10E+07 | 5.25E+07 | 99.1250% | 114.29 | 30 sec | F = 161 PRF = 100 Burst = 2000 |
| | | | | | | | | DTY = 50% Amp = 0.5 Offset = 0 |
| p46 | 1.00E−06 | 13 | 1.30E+07 | 3.25E+07 | 99.4583% | 184.62 | 30 sec | F = 161 PRF = 100 Burst = 2000 |
| | | | | | | | | DTY = 50% Amp = 0.5 Offset = 0 |
| p47 | 1.00E−06 | 24 | 2.40E+07 | 6.00E+07 | 99.0000% | 100.00 | 60 sec | F = 161 PRF = 100 Burst = 2000 |
| | | | | | | | | DTY = 50% Amp = 0.5 Offset = 0 |
| p48 | 1.00E−06 | 43 | 4.30E+07 | 1.08E+08 | 98.2083% | 55.81 | 60 sec | F = 161 PRF = 100 Burst = 2000 |
| | | | | | | | | DTY = 50% Amp = 0.5 Offset = 0 |
| p49 | 1.00E−06 | 18 | 1.80E+07 | 4.50E+07 | 99.2500% | 133.33 | 60 sec | F = 161 PRF = 100 Burst = 2000 |
| | | | | | | | | DTY = 50% Amp = 0.5 Offset = 0 |
| p50 | 1.00E−06 | 44 | 4.40E+07 | 1.10E+08 | 98.1667% | 54.55 | 180 sec | F = 161 PRF = 100 Burst = 2000 |
| | | | | | | | | DTY = 50% Amp = 0.5 Offset = 0 |
| p51 | 1.00E−06 | 34 | 3.40E+07 | 8.50E+07 | 98.5833% | 70.59 | 180 sec | F = 161 PRF = 100 Burst = 2000 |
| | | | | | | | | DTY = 50% Amp = 0.5 Offset = 0 |
| p52 | 1.00E−06 | 48 | 4.80E+07 | 1.20E+08 | 98.0000% | 50.00 | 180 sec | F = 161 PRF = 100 Burst = 2000 |
| | | | | | | | | DTY = 50% Amp = 0.5 Offset = 0 |
| p53 | 1.00E−08 | 24 | 2.40E+09 | 6.00E+09 | | | | |
| | | | | 4.75E+07 | 99.2083% | 126.32 | 10 sec | |
| | | | | 1.36E+06 | 99.9774% | 4417.18 | 30 sec | |
| | | | | 1.31E+06 | 99.9782% | 4585.99 | 60 sec | |
| | | | | 9.17E+07 | 98.4722% | 65.45 | 180 sec | |
| | | | | 7.08E+07 | 98.8194% | 84.71 | 10 sec | |
| | | | | 4.33E+07 | 99.2778% | 138.46 | 30 sec | |
| | | | | 7.08E+07 | 98.8194% | 84.71 | 60 sec | |
| | | | | 1.05E+08 | 98.2500% | 57.14 | 180 sec | |

Sample packets from P31 to p37 exhibit maximum destruction, identified as Fold Decrease. Sample packet p53 is control sample.
PRF: Pulse Repetition Frequency
CFU: Colony Forming Units

After ultrasound exposure treatment, the spores were placed in a pre-weighed, pre-numbered Eppendorf tube and the tubes were sealed. One tube was used for each treatment.

The tubes containing the spores were weighed again to obtain the final weight of the spores. Sterile milliQ water was added to each tube to obtain a stock solution for each treatment at a concentration of 10 mg/ml. Serial dilutions were then prepared from each stock solution from 10⁻¹ to 10⁻¹⁰. Based on previous experience with Bt, 400 μl of 10⁻⁶, 10⁻⁸, and 10⁻¹⁰ dilutions were plated onto 60 mm petri dishes containing 2% LB agar medium in a sterile laminar hood. The petri dishes were placed in an incubator at 28° C. The dishes were checked the following day for bacterial growth and any signs of contamination. No external contamination was found and four days later the number of spores that survived in each treatment was determined.

The number of bacterial spores that survived was determined by counting the number of colonies that grew (were alive) on each petri dish for each treatment. (Each live spore will produce one colony. The number of colony forming units (CFUs) per ml (CFU/ml) based on the dilution counted and the volume of the spore suspension applied to each plate was calculated (see Table 1).)

3. Results

The results demonstrated that treatments # 31 to 37 were the most effective at reducing the spore load by NCU transduction (see Table 1), although most of the treatments destroyed at least some of the spores. In particular, treatments #31 to 37 reduced the spore load by 4 orders of magnitude (7.54×10⁶ CFU/ml for # 31 and 4.75×10⁵ CFU/ml for # 35). In the control group (untreated; # 53), the concentration of spore load was 6×10⁹. FIG. 2(a) shows a sample of Bt (plated at a dilution of 10⁻⁶) prior to NCU irradiation, and FIG. 2(b) shows the same Bt sample after 1 minute of NCU irradiation.

4. Discussion

Even with only one minute of irradiation with non-contact ultrasonic treatment, it is possible to significantly destroy Bt (reduce the spore load by two orders of magnitude) at 100 and 161 kHz frequencies. Even the best technologies currently in use, such as electron beam radiation, reduce spore loads only by three orders of magnitude. This new and exciting methodology of NCU transduction has the potential to be used as a general germicide for the sterilization of medical and surgical equipment and food materials, in addition to destruction of anthrax spores and other highly toxic microbes that may be used as an agent of bioterrorism.

The present invention is further disclosed in the publication “Destruction of Bacterial Spores by Phenomenally High Efficiency Non-Contact Ultrasonic Transducers,” by K. Hoover, M. Bhardwaj, N. Ostiguy, and O. Thompson, Material Research Innovation, Vol. 6, pp. 291-295, 2002, incorporated herein by reference.

The present invention has been described with reference to specific details of particular parameters thereof. It is not intended that such details be regarded as limitations upon the scope of the invention except insofar as and to the extent that they are included in the accompanying claims.

The invention claimed is:

1. A method for destroying spores, germs, and the like, comprising:
(a) using a non-contact ultrasound device that generates enough power to transfer ultrasound energy in gas or ambient air without the use of a liquid medium; and
(b) irradiating the spores, germs, and the like with the transferred ultrasound energy.

2. The method according to claim 1, wherein the non-contact ultrasound device generates high power ultrasound energy, said high power ultrasound energy emitted in planar, point, or cylindrically focused ultrasonic beams.

3. The method according to claim 2, wherein the distance between the non-contact ultrasound device and the spores, germs, and the like is about 1.0 mm to 7.0 mm.

4. The method according to claim 3, wherein the spores, germs, and the like are irradiated with the non-contact device in the frequency range of about 50 kHz to 5 MHz.

5. The method according to claim 4, wherein the non-contact device is excited in pulsed mode by about 1000 bursts to 2000 bursts of about 0.10 V to 1.0 V, said excitation augmented with a power amplifier of about 50 dB to 55 dB, said excitation having a pulse repetition frequency of about 100 ms to 500 ms.

6. The method according to claim 4, wherein said excitation of the non-contact device is excited in a continuous wave mode having an amplitude of about 0.1 V that increases to about 0.2 V after about 1 to 3 minutes, said amplitude augmented with a power amplifier of about 50 dB to 55 dB.

7. The method according to claim 5, wherein said excitation of the non-contact device lasts about 1 to 5 minutes.

8. The method according to claim 6, wherein said excitation of the non-contact device lasts about 10 to 20 minutes.

9. The method according to claim 8, wherein the distance between the non-contact ultrasound device and the spores, germs, and the like is about 2.0 mm to 6.0 mm.

10. The method according to claim 9, wherein the distance between the non-contact ultrasound device and the spores, germs, and the like is about 3.0 mm to 5.0 mm.

11. The method according to claim 10, wherein the spores, germs, and the like are irradiated with the non-contact device in the frequency range of about 85 kHz to 185 kHz.

12. The method according to claim 11, wherein the spores, germs, and the like are irradiated with the non-contact device in the frequency range of about 100 kHz to 161 kHz.

13. The method according to claim 12, wherein the non-contact device is excited in pulsed mode by about 1000 bursts to 2000 bursts of about 0.25 V to 1.0 V, said excitation augmented with a power amplifier of about 50 dB to 55 dB, said excitation having a pulse repetition frequency of about 100 ms to 500 ms.

14. The method according to claim 13, wherein the non-contact device is excited in pulsed mode by about 1000 bursts to 2000 bursts of about 0.5 V to 0.7 V, said excitation augmented with a power amplifier of about 50 dB to 55 dB, said excitation having a pulse repetition frequency of about 100 ms to 500 ms.

15. The method according to any one of claims 3 to 14, wherein the spores are Bacillus anthracis.

16. A method for destroying spores, germs, and the like, comprising:
(a) using a high power generating non-contact ultrasonic device that produces planar, point, or cylindrically focused ultrasonic beams, said beams transferred in gas or ambient air without the use of a liquid medium;
(b) separating the non-contact ultrasonic device from the spores, germs and the like by a distance of about 3.0 mm to 5.0 mm;
(c) irradiating the spores, germs, and the like with the non-contact ultrasonic device in a frequency range of about 50 kHz to 5 MHz; and
(d) exciting the non-contact ultrasonic device in pulsed mode by about 1000 bursts to 2000 bursts of about 0.5 V to 0.7 V, wherein said excitation is augmented with a power amplifier of about 50 dB to 55 dB, said excitation having a pulse repetition frequency of about 100 ms to 500 ms, wherein said excitation lasts about 1 to 5 minutes.

17. A method for destroying spores, germs and the like, comprising:
(a) using a high power generating non-contact ultrasonic device that produces planar, point, or cylindrically focused ultrasonic beams, said beams transferred in gas or ambient air without the use of a liquid medium;
(b) separating the non-contact ultrasonic device from the spores, germs and the like by a distance of about 3.0 mm to 5.0 mm;
(c) irradiating the spores, germs and the like with the non-contact ultrasonic device in a frequency range of about 50 kHz to 5 MHz; and
(d) exciting the non-contact ultrasonic device in a continuous wave mode having an amplitude of about 0.1 V that increases to about 0.2 V after about 1 to 3 minutes, said amplitude augmented with a power amplifier of about 50 dB to 55 dB, wherein said excitation lasts about 10 to 20 minutes.

18. A method for destroying Bacillus anthracis spores, comprising:
(a) using a high power generating non-contact ultrasonic device that produces planar, point, or cylindrically focused ultrasonic beams, said beams transferred in gas or ambient air without the use of a liquid medium;
(b) separating the non-contact ultrasonic device from the spores, germs and the like by a distance of about 3.0 mm to 5.0 mm;
(c) irradiating the spores, germs and the like with the non-contact ultrasonic device in a frequency range of about 100 kHz to 161 kHz; and
(d) exciting the non-contact ultrasonic device in pulsed mode by about 1000 bursts to 2000 bursts of about 0.5 V to 0.7 V, wherein said excitation is augmented with a power amplifier of about 50 dB to 55 dB, said excitation having a pulse repetition frequency of about 100 ms to 500 ms, wherein said excitation lasts about 1 to 5 minutes.

19. A method for destroying Bacillus anthracis spores, comprising:
(a) using a high power generating non-contact ultrasonic device that produces planar, point, or cylindrically focused ultrasonic beams, said beams transferred in gas or ambient air without the use of a liquid medium;
(b) separating the non-contact ultrasonic device from the spores, germs, and the like by a distance of about 3.0 mm to 5.0 mm;
(c) irradiating the spores, germs, and the like with the non-contact ultrasonic device in a frequency range of about 100 kHz to 161 kHz; and
(d) exciting the non-contact ultrasonic device in a continuous wave mode having an amplitude of about 0.1 V that increases to about 0.2 V after about 1 to 3 minutes, said amplitude augmented with a power amplifier of about 50 dB to 55 dB, wherein said excitation lasts about 10 to 20 minutes.
